(19) United States
(12) Patent Application Publication
(10) Pub. No.: US 2014/0180406 A1
(43) Pub. Date: Jun. 26, 2014

(54) ACCOMMODATING INTRAOCULAR LENS

(71) Applicant: NOVARTIS AG, Basel (CH)

(72) Inventor: MICHAEL J. SIMPSON, ARLINGTON, TX (US)

(73) Assignee: NOVARTIS AG, Basel (CH)

(21) Appl. No.: 14/108,528

(22) Filed: Dec. 17, 2013

Related U.S. Application Data
(60) Provisional application No. 61/740,611, filed on Dec. 21, 2012.

(51) Int. Cl. A61F 2/16

(52) U.S. Cl. CPC A61F 2/1635
USPC 623/6.13

(57) ABSTRACT

A dynamic accommodative intraocular lens is provided in which a central lens portion of the intraocular lens undergoes dynamic change in curvature to adjust focus from distant objects to those nearby in response to natural accommodative actions of the patient's eye. The intraocular lens utilizes fluid movement from flexible fluid reservoirs defined along or adjacent haptic elements of the intraocular lens, which are engaged and compressed by the accommodating movements of the ciliary body of the patient's eye to cause expansion and flattening of the intraocular lens to adjust the optic power of the lens.

This application claims the priority of U.S. Provisional Patent Application No. 61/740611 filed on Dec. 21, 2012.

### FIELD OF THE INVENTION

The present invention relates generally to intraocular lenses (IOLs), and particularly to accommodating IOLs wherein the power of the lens can be dynamically adjusted in response to an accommodative stimulus.

### BACKGROUND OF THE INVENTION

A person's eye generally has an optical power determined by the optical power of the cornea and natural crystalline lens of the eye. In many people, this optical power can be diminished and/or change, especially with age, requiring correction by glasses or contact lenses. In addition, in some patients, the natural lens of the eye can become clouded, a condition commonly known as a cataract, due to the natural aging process, as well as effects of certain diseases such as diabetes. This clouding or cataract, as it worsens, can ultimately significantly adversely affect a patient's vision. Increasingly, intraocular lenses have been employed to replace such clouded natural lenses and to restore patients' vision. Prior IOLs primarily were monofocal (i.e., they focused light from distant objects onto the retina), to improve distance vision, but for nearer objects, the patient implanted with such monofocal IOLs often had to use reading glasses to correct or improve vision for close up or nearer objects. More recently, presbyopic accommodating IOL designs have been attempted with limited success.

One drawback with current IOL designs has been, however, the ability to design a lens that can provide reliable accommodation by the muscles of the eye that will be effective for a number of years following implantation, without requiring additional adjustments or replacements. As the ciliary muscles weaken and/or retract, the accommodative forces acting on the IOL can become lessened, thus reducing its effectiveness at correcting the vision of the patient. In addition, conventional IOLs are further often susceptible to posterior capsular opacification, which can also disrupt optical properties by clouding the lens and over time, requiring correction.

Accordingly, it can be seen that there is a need for an enhanced accommodative IOL, especially IOLs that address the foregoing problems of providing reliable accommodation over extended periods of time and address the difficulties of posterior capsular opacification, as well as various other related and unrelated problems in the art.

### SUMMARY OF THE INVENTION

Briefly described, the present invention generally relates to accommodating intraocular lenses (IOLs) adapted to be inserted into a patient's eye for adjusting the vision thereof. In one embodiment, the IOL can be implanted within the capsular bag of the patient's eye, replacing the natural crystalline lens thereof. The IOL generally will include a lens body formed from a flexible, hydrophilic or hydrophobic material, including various acrylic, silicone or hydrogel materials that are compatible with the human body. The lens body of the IOL can be formed as a flexible membrane or housing, including a base lens and a flexible or expandable optic membrane or center lens portion. An interior chamber generally is defined between the base lens and optic membrane and can receive an aqueous fluid or gel material, therein. At least one, and generally two or more haptic elements can extend radially outwardly from the lens body to an extent or position sufficient to be engaged by the ciliary body of the patient's eye during an accommodating action.

In one example embodiment, the haptics can be formed with flexible reservoirs at the distal ends thereof adapted to be received and seated within the sulcus portions of the ciliary body/muscle of the patient's eye. Each of the reservoirs generally will be filled with a fluid material similar to the fluid or gel material within the interior chamber of the lens body, with each reservoir in fluid communication with the interior chamber via ducts or channels formed through the haptic elements. As a result, movement of the ciliary body of the eye during accommodation, the reservoirs will be compressed, causing the fluid or gel material therein to move into the interior chamber of the lens body. The introduction of the additional fluid material into the interior chamber of the lens body in turn will cause the optic membrane to be urged forwardly, expanding or bulging approximately in the center thereof to adjust the optical power of the lens.

Alternatively, the haptic elements themselves can be formed as fluid reservoirs, with the distal ends of the haptic elements directly engaging the ciliary body adjacent the sulcus portions thereof. As the ciliary body is accommodated, the haptic elements will be compressed, causing the fluid therein to flow into the interior chamber of the lens body, and causing the outer lens portion to bulge or expand forwardly. In addition, rather than flow into the interior chamber of the lens body, the fluid material of the reservoirs can be urged/directed against the sides of the flexible membrane defining the lens body, causing the sides of this flexible membrane to compress or flex inwardly and displace the fluid material within the interior chamber so as to cause the optic membrane to bulge or expand forwardly.

In another embodiment, a second IOL also can be provided in front of the base or first IOL that is received within the capsular body. The second IOL can include one or more flexible reservoirs located within the sulcus portions of the ciliary body of the patient's eye, while the first IOL can be a conventional IOL having one or more haptic elements extending radially therefrom and toward the ciliary body of the patient's eye. The second lens can be used to provide part or most of the power correction by engagement of its haptic elements by the ciliary body during accommodating movement thereof, and can help inhibit and/or enable correction of posterior capsular opacification.

Various objects, features and advantages of the present invention will become apparent to those skilled in the art upon a reading of the following description, when taken in conjunction with the accompanying drawings.

### BRIEF DESCRIPTION OF THE DRAWINGS

FIG. 1A is a perspective illustration of a first embodiment of the accommodating IOL according to the principles of the present invention.

FIG. 1B is a side elevational view of the accommodating IOL of FIG. 1A.

FIG. 2 illustrates a second embodiment of the accommodating IOL according to the principles of the present invention.

FIG. 3 is a perspective view illustrating yet another embodiment of the accommodating IOL according to the principles of the present invention.

Those skilled in the art will appreciate and understand that, according to common practice, the various features of the drawings discussed below are not necessarily drawn to scale, and that dimensions of various features and elements of the drawings may be expanded or reduced to more clearly illustrate the embodiments of the present invention described herein.

### DETAILED DESCRIPTION

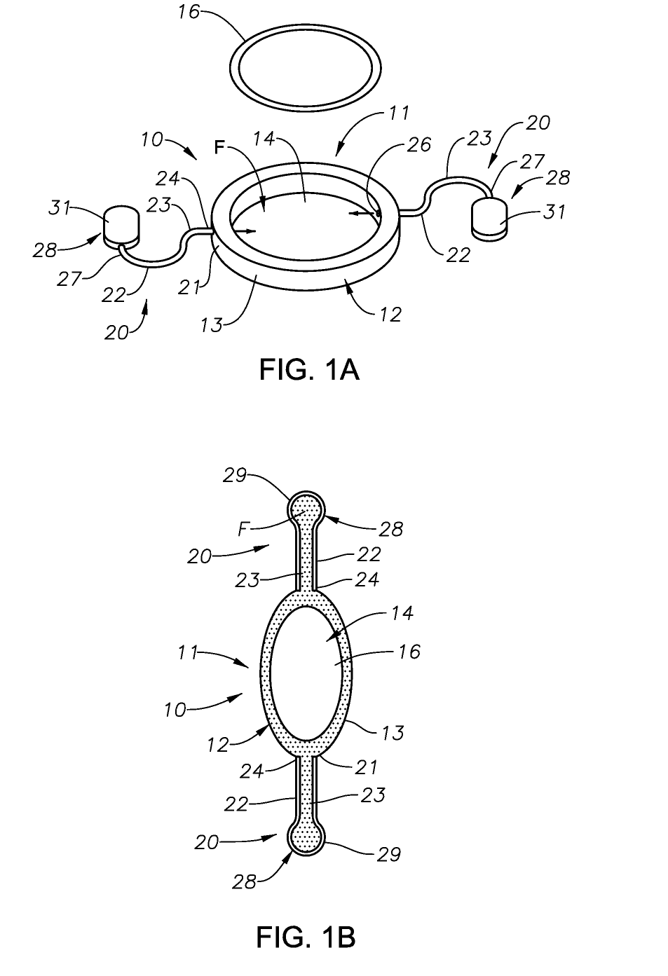

FIGS. 1A-3 illustrate various embodiments of accommodating intraocular lenses (IOLs) according to the principles of the present invention. The IOLs are designed to be inserted into the eye of a patient and function in response to the natural accommodative forces of the patient's eye to adjust the optical power of the lenses, and therefore the vision of the patient, to improve focus and clarity of the patient's vision as to nearer or distant objects. In each of the illustrated embodiments, the IOLs generally can be formed of a soft, flexible acrylic, silicone or hydrogel material, which can include hydrophyllic as well as hydrophobic acrylic, silicone or hydrogel lens materials as will be understood in the art. For example, the IOLs can be formed from AcrySof® acrylic intraocular lens materials from Alcon Laboratories, Inc. The present IOUs further can contain or be filled with a transparent, aqueous fluid material such as a liquid, gel or flexible solid material that additionally can have a refractive index higher than that of the natural aqueous fluid of the patient's eye in which the IOL is implanted.

FIGS. 1A-1B illustrate a first example embodiment of an accommodating intraocular lens 10 according to the principles of the present invention. In this embodiment, the lens body 11 of the IOL 10 generally will be implanted within the posterior chamber P (FIG. 1C) of the patient's eye, located within the capsular bag and replacing the natural crystalline lens of the eye. As shown in FIGS. 1A-1B, the IOL 10 generally includes a lens body 11 that can be formed as a substantially hemispherically shaped housing or flexible membrane, indicated at 12. The lens body can include a base lens portion 13 defining a base optic power for the IOL and having an interior chamber 14 formed therein, and a flexible, expandable optic membrane or central lens portion 16 extending along or across the base lens so as to cover and/enclose the interior chamber 14. As indicated in FIG. 1B, the lens body 11 can be formed as a substantially unitary one-piece structure, or, alternatively, can be formed in separate pieces or sections as indicated in FIG. 1A with the optic membrane 16 being applied to the base lens 13 and attached or bonded thereto by plasma bonding, adhesive attachment or other attachment means.

The interior chamber 14 further generally will be filled with a transparent aqueous fluid material F such as a liquid, gel or flexible solid material. As noted, the fluid material typically has a refractive index higher than that of the natural aqueous fluid of the patient's eye so as to provide the IOL with a desired optic power in addition to the base power of the base lens 13. The fluid material substantially fills the interior chamber 14 of the IOL such that changes in the volume and/or pressure of the fluid material within the interior chamber will cause the fluid material to move and bear against the optic membrane 16. In response, the optic membrane will flex and expand/bulge or contract/flatten with the movement of the fluid material against and away from the optic membrane, adjusting the visual power of the IOL for focusing on nearer or distant objects.

As indicated in FIGS. 1A-1B, haptic elements 20 generally will project radially away from a peripheral side edge 21 of the base lens portion 13 of the lens body 11. While FIGS. 1A-1B illustrate the use of a pair of radially extending haptic elements located on opposite sides of the lens body, it will be understood that greater or fewer haptic elements also can be utilized, as well as the use of different haptic element configurations. As indicated in FIGS. 1A and 1B, each of the haptic elements 20 generally can include elongated, flexible body portions 22 that extend away from the lens body and define fluid ducts or channels 23 therealong The haptic elements 20 further generally will be formed from the same or a similar flexible, hydrophyllic or hydrophobic materials from which the lens body is formed, and can be integrally formed with the lens body 11 or can be separately formed and attached to the peripheral side edge 21 of the lens body.

The haptic element body portions 22 also generally will include a first or proximal end 24 that is located along the peripheral side edge 21 of the lens body and can be in open fluid communication with the interior chamber 14 of the lens body via ports or openings 26 (FIG. 1A). The second or distal ends 27 of the haptic element bodies further can include flexible fluid reservoirs 28. As indicated in FIGS. 1A and 1B, such flexible reservoirs 28 can be formed as expanded or enlarged end portions 29 of the body portions of the haptic elements (as shown in FIG. 1B), or alternatively, can be formed as compressible bladders 31 (FIG. 1A) attached to the distal ends 27 of the haptic element body 22.

The flexible reservoirs 28 and/or the fluid ducts 23 of the haptic elements 20 further generally will be filled with or contain a fluid material such as a liquid, gel or flexible solid material that is the same as or is compatible with the fluid material F with which the interior chamber 14 of the lens body 11 is filled. The ports or openings 26 formed through the base lens portion 13 of the lens body 11, and through which the first or proximal ends 24 of the haptic elements communicate with the interior chamber of the lens body, enable transfer of the fluid material between the fluid reservoirs of the haptic elements and the interior chamber of the lens body as the haptic elements are engaged and acted upon by the muscles of ciliary body (FIG. 1C) of the patient's eye during an accommodative operation of the patient's eye. Alternatively, rather than the fluid flowing into the interior chamber 14 directly, the fluid from the fluid reservoirs 28 will be urged against the flexible side edges of the lens body, causing the sides of the lens body to be compressed and urge the fluid material within the interior chamber against the optic membrane.

The accommodative IOL 10 generally will be implanted within the capsular bag of the patient's eye as a replacement for the natural crystalline lens of the patient's eye. During such an operation, the natural crystalline lens generally will be removed from the capsular bag via an incision into which the IOL will then be implanted into the capsular bag. The IOL can be folded or rolled into a more compact configuration and thereafter injected via a plunger into the capsular bag. In addition, the haptic elements will be extended and placed into sulcus portions S of the patient's eye. As a result, the flexible reservoirs 28 formed or attached at the distal ends 27 of the haptic elements 20 of the accommodating IOL 10 will be received and supported within the sulcus portions of the patient's eye.

Thereafter, as the muscles of the ciliary body of the patient's eye contract about the flexible reservoirs 28 during accommodation of the eye, the flexible reservoirs generally will become compressed, causing the fluid material contained therein to flow along the fluid ducts 23 and into the interior chamber 14 of the lens body 11. The influx of fluid material into the interior chamber accordingly creates an increase in volume of the fluid material therein, which correspondingly increases the pressure that the fluid material exerts on the optic membrane 16. In response, the optic membrane of the IOL is caused to expand or bulge forwardly and away from the base lens 13. Such expansion accordingly results in a change of the curvature and thus the optical power provided by the optic membrane to enable adjustment of the patient's vision to focus more clearly on nearer objects. Thereafter, as the muscles of the ciliary body relax or contract, the pressure exerted on the flexible reservoirs in the sulcus portion is relaxed and the fluid material is permitted to flow from the interior chamber of the lens body back into the flexible reservoirs 28 via the fluid ducts 23 of the haptic elements 20, thus reducing the pressure of the fluid material acting upon the optic membrane. In response, the optic membrane accordingly contracts and moves back to a generally flattened, relaxed, non-distended position to enable the patient's eye to refocus on nearer objects with greater clarity.

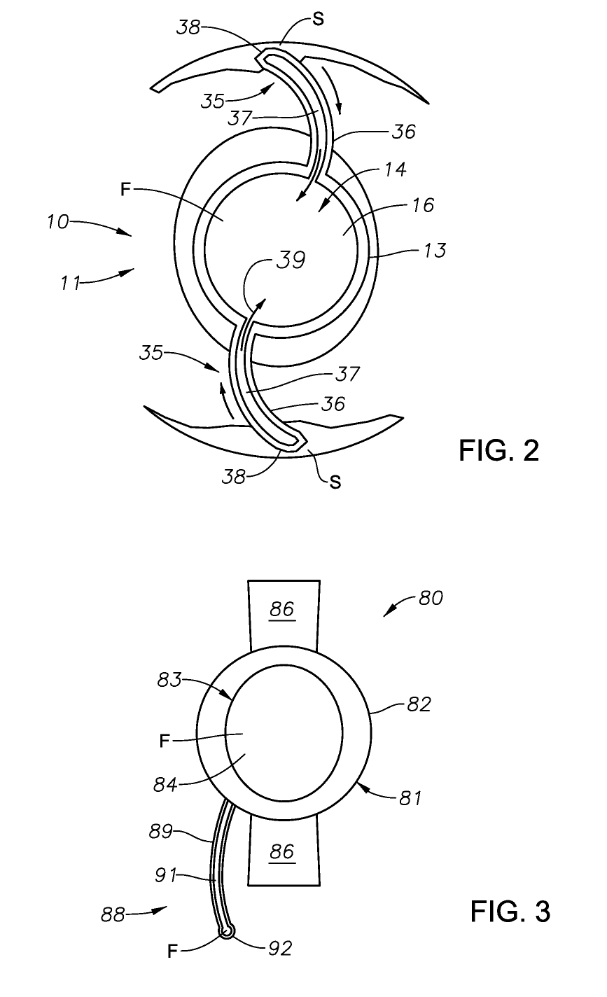

FIG. 2 illustrates a further alternative embodiment of the accommodative IOL 10, in which haptic elements 35 project outwardly from the base lens 13 of the lens body 11. These haptic elements 35 generally can be provided with an expanded or enlarged haptic body portion 36 so as to define the flexible reservoirs having elongated fluid chambers 37 extending therealong and filled with fluid material F. The distal ends 38 of the haptic elements 35 generally will project toward the ciliary body adjacent or extending into the sulcus portions of the patient's eye so that the distal ends of the haptic elements are supported therein and thus substantially fix the position of the intraocular lens within the patient's eye. As the muscles within the ciliary body of the patient's eye expand during accommodation, the haptic elements generally will be urged inwardly toward the lens body, causing compression of the haptic elements. In response, the fluid material contained within the fluid chambers 37 of the haptic elements 35 will be displaced and urged into the interior chamber 14 of the lens body as indicated by arrows 39. The resulting increase in the volume of the fluid material within the interior chamber of the lens body correspondingly increases the fluid pressure acting against the optic membrane 16, causing the optic membrane to bulge or expand outwardly or forwardly to adjust the vision of the patient.

FIG. 3 illustrates still another additional alternative embodiment of an IOL according to the principles of the present invention. In this embodiment, the IOL 80 generally comprises a lens body 81 having a base lens 82 with a flexible transparent optic membrane or center lens portion 83 extending across and substantially covering the base lens 82. An interior fluid chamber 84 is defined between the base lens 82 and optic membrane 83 and is substantially filled with a transparent, aqueous fluid material. The fluid material, as discussed above, typically can have a refractive index that is higher than the natural aqueous fluid of the patient's eye, with the overall power of the lens being adjusted by adjusting the shape of the optic membrane in response to accommodative forces of the patient's eye. The optic membrane and base lens can be integrally formed, or can be formed as separate elements that are attached or adhered together such as by plasma bonding, adhesion or other known attachment methods, so as to define an inflatable or partially inflatable IOL.

A flexible reservoir 88 can be connected to the lens body 81 and extends tangentially away from the lens body. The flexible reservoir 88 generally includes an elongated body 89 having a fluid passage or channel 91 defined therethrough, and terminates at a distally located compressive reservoir portion 92. The flexible reservoir 88 generally will be filled with fluid material F (FIG. 5B) so as to provide an additional supply or reservoir of the fluid material for filling the central interior chamber 84 of the IOL 80. The body 89 of the fluid reservoir 88 further can be formed separately from or can be at least partially formed with one of the haptic elements 86, extending along at least a portion of the haptic element. As shown in FIG. 5B, the reservoir body additionally will extend downwardly radially outwardly from the lens body with the compressive portion 92 of the distal end of the fluid reservoir body being received and seated within a sulcus portion S of the ciliary body of the patient's eye.

When the patient's eye thereafter disaccommodates, so as to view a more distant object, the ciliary body generally will relax, relieving pressure on the fluid reservoir. This in turn will provide a reduction in the pressure of the fluid material within the interior chamber of the IOL, reducing the thickness of the IOL to accordingly reduce the lens power of the IOL. As an alternative, the optic membrane can be of a reduced size or diameter so that only a central region of the lens will change shape to increase power during accommodation.

The foregoing description generally illustrates and describes various embodiments of the present invention. It, however, will be understood by those skilled in the art that various changes can be made to the above-discussed construction without departing from the spirit and scope of the present invention as disclosed herein, and it is intended that all matter contained in the above description or shown in the accompanying drawings shall be interpreted as illustrative and not in a limiting sense. Furthermore, the scope of the present disclosure shall be construed to cover various modifications, combinations, alterations, etc. of the above-described embodiments and shall be considered to be within the scope of the present invention. Accordingly, various features and characteristics of the present invention as discussed herein may be selectively interchanged and applied to other illustrated and non-illustrated embodiments of the invention.

1. An accommodating intraocular lens for implantation into a patient's eye comprising:
An inflatable lens body having a base lens and an optic membrane and defining an interior chamber therein, said interior chamber containing a volume of a fluid material therein;
at least one haptic element extending radially from said lens body and including a flexible reservoir formed adjacent an end portion thereof and adapted to be engaged by a portion of a ciliary body of the patient's eye; and
a fluid material received within said flexible reservoir of said at least one haptic element;
wherein as the ciliary muscle is moved during accommodation, said flexible reservoir becomes compressed in a ciliary sulcus of the eye transferring at least a portion of said fluid material therein to said interior chamber of said lens body, increasing the volume of fluid material therein, which causes said lens body to change shape and thus vary a power of the intraocular lens.

2. The accommodating intraocular lens of claim 1, wherein said flexible reservoir body of said at least one haptic element is received within a sulcus portion of the ciliary body.

3. The accommodating intraocular lens of claim 1 and wherein said at least one haptic element further comprises a first haptic element and a second haptic element on an opposite side of said lens body from said first haptic element.

4. The accommodating intraocular lens of claim 3, wherein said flexible reservoir comprises a distal end portion of said at least one haptic element received within a sulcus portion of the ciliary muscle.

5. The accommodating intraocular lens of claim 1, and further comprising a second lens positioned posterior to said lens body within a capsular bag of the patient's eye.

6. The accommodating intraocular lens of claim 5, wherein said flexible reservoir comprises a distal end portion of said at least one haptic element that engages a sulcus portion of the ciliary muscle.

7. The accommodating intraocular lens of claim 1, wherein said lens body is received within an anterior chamber of the eye, supported by said at least one haptic element above an iris portion of the eye, and wherein said flexible reservoir comprises a body that extends distally away from said end portion of said at least one haptic element and a distally located compressive portion that contacts the ciliary muscle within a posterior chamber of the eye.
